(19) United States
(12) Patent Application Publication
(10) Pub. No.: US 2019/0391155 A1
(43) Pub. Date: Dec. 26, 2019

(54) LABELING OF ALUMINOSILICATES

(71) Applicant: Glock Health, Science and Research GmbH, Deutsch Wagram (AT)

(72) Inventor: Gaston GLOCK, Velden (AT)

(21) Appl. No.: 16/453,414

(22) Filed: Jun. 26, 2019

(30) Foreign Application Priority Data
Jun. 26, 2018 (EP) 18179915.6

(51) Int. Cl. G01N 33/58; G01N 1/30; G01N 33/84; G01N 21/64; G01N 33/24

(52) U.S. Cl. CPC G01N 33/582; G01N 1/30; G01N 33/84; G01N 2021/6439; G01N 33/24; G01N 2001/307; G01N 21/6428

(57) ABSTRACT

Labeling and detection of clinoptilolite and other zeolites and aluminosilicates by means of lumogallion fluorescence reaction in paraformaldehyde-fixed animal and human cell cultures and tissue samples after administration of the mineral or in mineralogical-geological samples themselves.

The invention relates to a specific labeling of aluminosilicates, in particular the specific detection and labeling of zeolite particles in different matrices in accordance with the introductory part of Claim 1.

Due to their diverse use, the definite detection of aluminosilicates in biological samples is of crucial importance. For example, within the agricultural sector, aluminosilicates are introduced into the soil as a natural fertilizer. In livestock breeding and fattening, alumosilicates are used to maintain health and promote weight gain of the animals. For use in and/or with humans, alumosilicates are introduced in the same manner and remain in either original or modified form (e.g., summarized by Mumpton, 1999). Despite the widespread use of aluminosilicates, their detection is highly demanding and difficult due to high technical requirements.

In general, aluminosilicate compounds can be detected directly via X-ray diffraction (XRD, specific but with relative insensitivity) or indirectly via electron microscopy (nonspecific).

The use of aluminum-specific staining methods in biological research involves the use of chromophores such as hematoxylin, eriochrome cyanine R, aluminon and azide solochromic azurin. However, as these are relatively nonspecific and show only low spatial resolution, they could not be used for studies of the cellular distribution of aluminum ions (Eticha et al., 2005). For this purpose, the fluorophores morin (2′,3,4′,5,7-pentahydroxyflavon) and lumogallion (5-chloro-3-(2,4-dihydroxyphenylazo)-2-hydroxybenzenesulfonic acid) were used, both of which are suitable for the detection of aluminum (Illes et al., 2006), with morin being at least partially less specific and sensitive compared to lumogallion (Kataoka et al., 1997). An example for this is that aluminosilicate particles embedded in gelatin or in intestinal tissue could not be detected with Morin (Powell, 2002).

The histochemical stains solochromic azurin, solochromic cyanine, and aluminon were tested by Powell for the direct staining of aluminosilicate particles and found to be unsuitable (Powell, 2002).

The localization of aluminum-ion compounds through the use of lumogallion in various biological issues over the last decades comprises an extremely broad range of topics—in contrast to aluminum bound in aluminosilicate particles —:
1. Water:
   One of the first applications of lumogallion was the identification of dissolved aluminum in water samples (e.g., Hydes and Liss, 1976, Caschetto and Wollast, 1979, Jonasson, 1980, He et al., 1997, Ren et al., 2001), which was first published by Nishikawa and colleagues (Nishikawa et al., 1967 and 1968, Shigematsu et al., 1970).
   He et al., 1997, tested the specificity of lumogallion on ions (at a concentration of 10 ppm per element) which can occur in natural water: Fe³⁺, Ca²⁺, Mg²⁺, Cu²⁺, and Zn²⁺, and confirmed the selectivity of the reaction of lumogallion with Al³⁺ (He et al., 1997). Mirza and colleagues embedded various forms of metal ions (Al(III), Ca(II), Cu(II), Fe(III), Mg(II), Zn(II)) in agarose and made sections of them, which after lumogallion staining were analyzed under a fluorescence microscope. Using the trivalent aluminum sections, they found only one fluorescent reaction with lumogallion (Mirza et al., 2016).
   Both T. R. Crompton and G. Sposito cite Hydes and Liss's 1976 publication in their books and conclude that “All forms of aluminum in filtered water may be detected except when the aluminum occurs in stable mineral structures, such as clay particles, small enough to pass through the filter.” (Crompton, 2015), and “Lumogallion does not react with suspended clay minerals, but the method does seem to determine small quantities of absorbed aluminum on kaolinite.” (Sposito, 1995).
   In J. J. Powell's paper, published in 2002, the inability to stain aluminosilicates using histochemical staining methods (solochrome azurin, solochrome cyanine, aluminon, Morin), as opposed to aluminum hydroxide and aluminum phosphate is rationalized as follows: “Both aluminium hydroxide and aluminium phosphate are insoluble at physiological pH and are well bound forms of the metal. However, sufficient aluminium ions are available for chelation at the surface of these species to give strong, positive reaction with the stains used here. In contrast, aluminosilicates, which are especially well bound forms of aluminium, clearly have insufficient available aluminium ions at their surfaces for detection by conventional histochemical staining and other methods were therefore sought.” J. J. Powell concludes: “Thus in conclusion it was not feasible to demonstrate aluminium histochemically when it was bound to aluminosilicates.” (Powell, 2002).
2. Plants:
   More than 40% of arable land worldwide has a very acidic pH (<5), which releases the aluminum compound Al³⁺ from soil containing clay, which in turn has a direct effect on the plants, often resulting in drastic crop failures (Sivaguru et al., 2013). Therefore, research into the effects of aluminum on plants worldwide is of particular interest. Sivaguru et al., 2013, described a resistance mechanism of a millet species (Sorghum bicolor) to aluminum, which they detected in the roots of the plant by means of the lumogallion fluorescence reaction.
   For example, within the plant physiology studies, fluorescence lifetime imaging (FLIM) analysis has been used to live-track the uptake of Al³⁺ into Arabidopsis thaliana root cells (Barbourina and Rengel, 2009). In 2000, Silva and colleagues published a paper on soybeans (Glycine max L. Merr.) which had been treated with Al³⁺ and were able to localize the distribution of aluminum ions in the root tip using lumogallion reaction.
   Kataoka et al., 1997, studied the viability and growth recovery in tobacco plants (Nicotina tabacum L.) and soybeans (Glycine max (L.) Merr. cv. Tsurunoko) following the addition of aluminum chloride AlCl₃. They tested various aluminum-staining substances based on colorimetric methods (aluminon, hematoxylin, pyrocatechol) or fluorescence staining (Lumogallion and Morin) and confirmed the use of lumogallion as the most sensitive detection method using epifluorescence and confocal microscopy to analyze the content on soluble aluminum ions (Kataoka et al., 1997). The effect of AlCl₃ on photosystem II was also analyzed in tobacco plants using a lumogallion reaction (Li et al., 2012).
3. Rock samples:
   Flow-injection analyses (FIA) with fluorescence detection of aluminum from soil samples were performed after separation of the individual water-soluble aluminum species using ion exchangers by means of lumogallion (Yamada et al., 2002).
   The resolution of both well and poorly crystallized kaolinites was the research subject in the 1999 publication of Sutheimer and colleagues. Using high-performance cation exchange chromatography, which allows for detection of free Al³⁺ as well as complexed aluminum to a minimum concentration of 7 nM (this method not being suitable for detecting polymeric aluminum oxyhydroxides), soluble aluminum was identified in the eluates after gradient elution using lumogallion (Sutheimer et al., 1999).
      Montmorillonite, which was incubated in artificial lung fluid and partially resolved by it, was used to simulate the degradation of inhaled particles in the lung. Analysis of the total aluminum content of the montmorillonite in solution was subsequently carried out using the fluorescence measurement method on the artificial lung fluid, also using lumogallion (Ramos Jareño, 2013). This publication did not describe a direct coloring of aluminosilicates either.
4. Animal and human:
   The detection and quantification of aluminum ions in human blood and urine samples using HPLC were published by Lee et al., 1996.
   Detection of the distribution of aluminum in bone and lung tissue of Wistar rats after intraperitoneal administration of potassium aluminum sulfate and aluminum hydroxide in the diet was achieved using a confocal laser microscope after fixation and embedding of the tissue samples in methyl methacrylate casting resin and subsequent staining of aluminum ions in the histological sample with lumogallion (Uchiumi et al., 1998).
   In 2005, Zhou and Yokel published a paper in which a cell culture model of the gastrointestinal absorption of ²⁷Al as an ion, citrate, maltolate, fluoride, or hydroxide was investigated and in which aluminum was visualized during confocal microscopic analysis using lumogallion.
   Furthermore, staining of aluminum-containing substances in living (Mile et al., 2015) and fixed cells as well as histological thin-section specimens was achieved using fluorescence microscopy (Mold et al., 2014; 2016, and Mirza et al., 2016; 2017). In this case, aluminum oxide hydroxide ions (AlO(OH)), as used in adjuvants, were identified in a monocyte cell line under physiological conditions (Mile et al., 2015) and after fixation of the cells (Mold et al., 2014 and 2016). Furthermore, detection of aluminum ions was achieved in Histo-Clear dewaxed histological specimens of Alzheimer's patients with lumogallion (Mirza et al., 2016 and 2017).
   In 2014, Klein and colleagues published the results of the study of human seminal fluid on aluminum. This group also used lumogallion for parts of their experiments (Klein et al., 2014).

Despite the widespread use of lumogallion, there has been no possibility of labeling any aluminosilicate particles with lumogallion until now.

Patents describing the use of lumogallion are, for example:
Aya Ohkubo, Osamu Shirota, Makoto Sato, Hajime Yoshimura, inventors; 2000.
Chelating reagent and measuring aluminum and measuring method.
Application: Sep. 12, 2000. US 2004/0101968 A1. May 27, 2004.
Using chelator 2,2′-dihydroxy-azobenzene, which on the one hand binds to lumogallion and to the aluminum-containing sample on the other hand, the aluminum content is determined using chromatography without affecting other substances present in the sample. This method comprises e.g. the following samples: biological materials, food, beverages, drinking water, reagents, pharmaceuticals, river water, lake water, seawater, and soils and is particularly suitable for measuring the aluminum concentration in the blood of dialysis patients in the context of laboratory medical examinations as well as for determining the aluminum concentration in pharmaceutical products.
Essential for determining the aluminum content of the sample in this case is that a reaction solution containing the aluminum from the sample is present. If there is no liquid sample present for aluminum analysis, it must be prepared beforehand (e.g., by extraction, solubilization, etc.) including deproteinization in biological samples.
Daido Ishii, inventor, 2001.
Method for determination of aluminum.
Application: Dec. 19, 2001. US20040259262 A1. Dec. 23, 2004.
The measurement of the aluminum content using liquid chromatography (HPLC) can be carried out at a buffered pH of >6.0 and room temperature within 2 min, and not as usual at more acidic pH-levels with incubation of the sample at 80° C. for 20 min.
In this invention, the aluminum content of the sample, which may for example be a pharmaceutical, vegetable or animal tissue, health food, drinking water, tea, a cosmetic, an alcoholic beverage, tap water, a water sample, seawater, lake water, river water, industrial waste water, process water, a research reagent, industrial raw material, an antibody or vaccine from an antigen, serum, urine, plasma, blood, bodily fluid of human or animal origin, sweat, tear fluid, ascites fluid or amniotic fluid, is also measured in a solvent.

Analysis of fluorescent dyes using fluorescence microscopy often requires the use of mounting media, which prevent fading of the substances.

Chemical solutions to the problem of photobleaching have been sought, especially in the context of and beginning with the use of immunofluorescence detection in the early 1940s, using fluorescent secondary antibodies (Coons et al., 1941, 1942, 1950). G. D. Johnson and colleagues, through the use of p-phenylenediamine (PPD) in buffered glycerin as mounting medium, were able to achieve a significant retardation of bleaching during the evaluation of a fluorescent sample using fluorescence microscopy (Johnson and Nogueira Araujo 1981, Johnson et al., 1982). Before that, mounting media often consisted only of a 1:9 mixture of PBS (phosphate buffered saline) or TRIS (trisodium phosphate) with glycerol (Huff et al., 1982; Valnes and Brandtzaeg, 1985), which was unlikely to prevent rapid bleaching and made photographic documentation of the evaluations almost impossible (Huff et al., 1982). In contrast, specimens prepared with PPD-containing mounting medium proved stable not only for at least one week when stored at 4° C. before onset of gradual fading but were also observable at much higher intensity and hence with better morphological detection using fluorescence microscopy (Huff et al., 1982). Furthermore, the use of PPD showed an increase of fluorescence activity while having no influence on antigen-antibody binding (Platt and Michael, 1983).

PPD is contained, for example, in VECTASHIELD Antifade Mounting Medium (Vector Laboratories, USA) (Longin et al., 1993; Cordes et al., 2011).

Other additives in mounting media include, but are not limited to, for example, n-propyl gallate (NPG) and 1,4-diazobicyclo(2,2,2)octane (DABCO) (e.g. Valnes and Brandtzaeg, 1985; Longin et al., 1993; Florijn et al, 1995), which, however, require very high concentrations to be effective (Longin et al., 1993), as well as ascorbic acid (AA), 6-hydroxy-2,5,7,8-tetramethylchromane-2-carboxylic acid (Trolox), mercaptoethylamine (cysteamine) and cyclooctatetraene (Cordes et al., 2011).

The object of the present invention is to provide fast, stable, non-toxic and cost-effective labeling and detection of aluminosilicates themselves, such as those e.g. in histological sections without necessary prior pretreatment such as dewaxing, in lavages, geological samples (mineral particles, rocks, etc.) and cell culture preparations.

According to the invention this object is achieved by a method having the features of the characterizing part of claim 1. In other words, by using:
Lumogallion (5-chloro-3-(2,4-dihydroxyphenylazo)-2-hydroxybenzenesulfonic acid) and
Mounting medium with para-phenylenediamine (1,4-diaminobenzene) in glycerol.

Such a surprisingly usable mounting medium is, for example, VECTASHIELD Antifade Mounting Medium, Vector Laboratories, USA.

The invention thus provides a method for the specific and stable labeling of zeolites, in particular of clinoptilolite, in cell cultures and tissue sections, but also of the particles themselves, which are characterized by their rapid, highly reproductive, cost-effective and technically uncomplicated handling.

The attached drawing represents in:

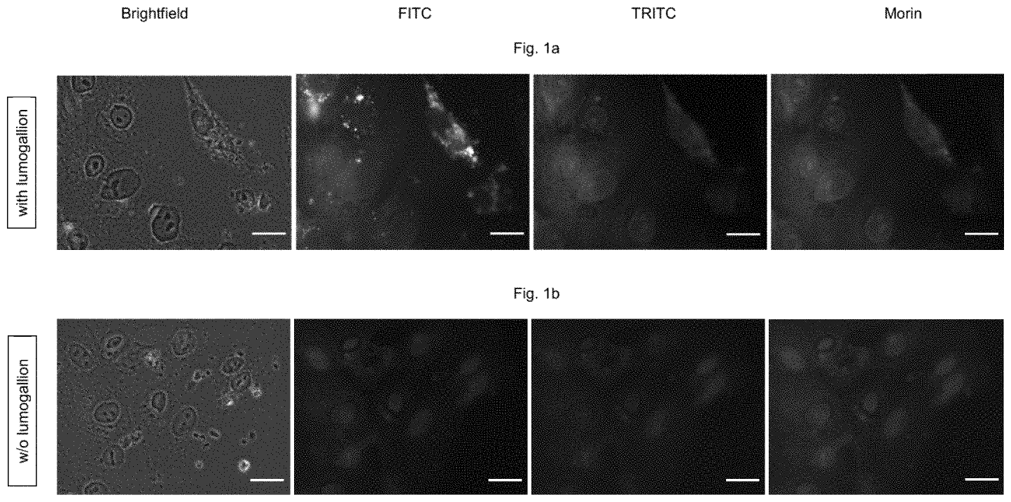

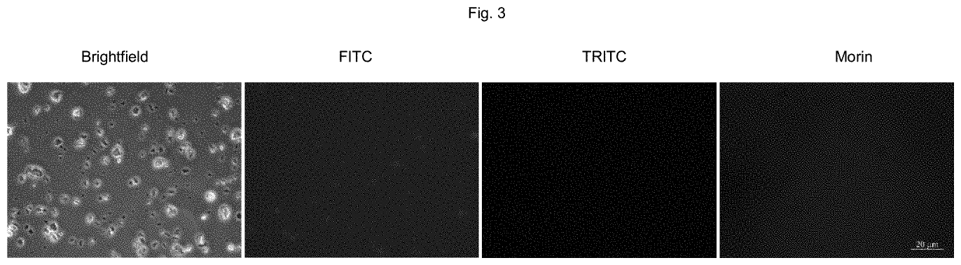

FIGS. 1, 2, 4, 5, and 6 examples for the staining and in FIG. 3 an example without evaluable staining, and are explained in more detail below.

More specifically, it is advantageous for the application of the labeling of the alumosilicates alone or in biological specimens by means of lumogallion, both for the duration and the incubation temperature, as well as the concentrations, to keep pH values of the buffers and solutions used as well as the particle number in accordance with the following schedule:
a. Zeolites or other aluminosilicates: 10 μg/cm²
b. Diameter of particle size: approx. 1-60 μm
c. Lumogallion solution: 10-200 μM in 50 mM HEPES buffer pH 7.2 or 100-200 μM in 20-50 mM acetate buffer, pH 4.0.
d. Incubation period: 2 h-24 h
e. Incubation temperature: 20° C.-37° C./50° C.-80° C.

On an experimental scale, the fluorescence reaction may be carried out at (room) temperature of up to 37° C. for 12 to 24 hours (“overnight”) as well as at 50° C. to 80° C. within 2 to 4 hours.

Since labeling using lumogallion is known to be non-toxic, it is also possible for the experimenter to handle the solutions and the process in a comfortable and unproblematic manner while adhering to all protective measures required for working in a laboratory and with the required substances.

### DETAILED DESCRIPTION

### Cell Culture Monolayers:

In paraformaldehyde-fixed cell cultures, which were previously treated with aluminosilicates or aluminosilicates in mixtures with non-aluminosilicates, aluminosilicates can be definitely detected.
Any autofluorescence of the cells is surprisingly reduced by treatment with lumogallion and the labeled particles are unmistakably recognizable.
A particular advantage of this specific labeling of aluminosilicate particles using lumogallion is the surprisingly high stability of fluorescence, which can be further extended using an antioxidative mounting medium, so that even after several days to months of storage at 4° C. the samples remain analyzable under a microscope without significant loss of quality. FIGS. 1 and 5, as well as 6b depict several examples of this application.

### Tissue Samples:

Histological sections of paraffin-embedded, zeolite-containing samples (with a thickness of 5 μm-25 μm) may surprisingly also be stained with a buffered lumogallion solution without prior dewaxing.
As with cell culture monolayers, autofluorescence of the tissue layer is also suppressed (“quenched”) and the zeolites become specifically visible.
A peculiarity of this method lies in the surprisingly high stability of the labeling and physicochemical integrity of the fluorophore (lumogallion) after fixation and embedding, which can additionally be carried out using an antioxidant mounting medium. This counteracts any possible bleaching of the fluorophore by the excitation light during analysis using fluorescence microscopy (“photobleaching”). It was discovered that the samples are observable for a longer period of time without significant loss of fluorescence under the microscope and that they can be stored refrigerated at 4° C. (for several days to months).
FIG. 5 shows an analysis of samples which had been stored for 8 weeks in a refrigerator at 4° C. after lumogallion staining and whose fluorescence label is still clearly delineated and intensively bright after this period without any significant loss of quality.

### Mineralogical Geological Samples:

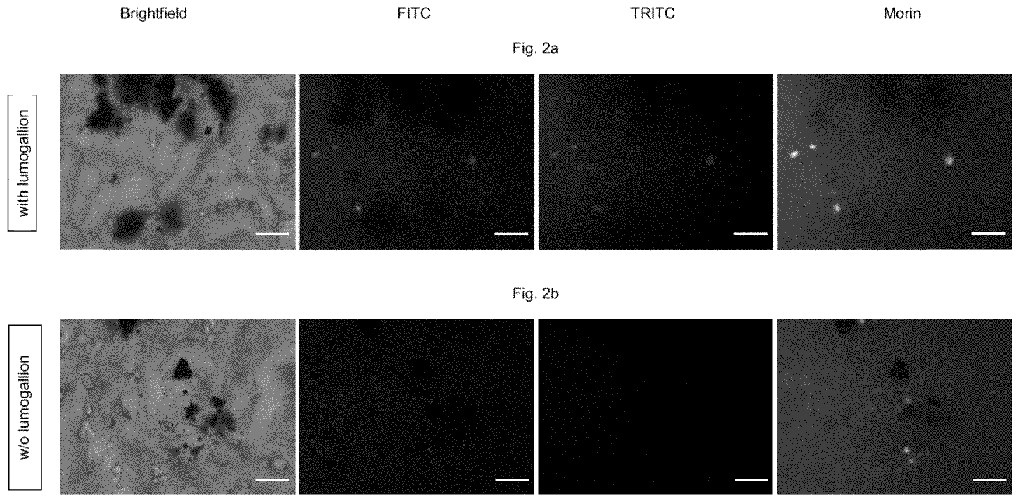

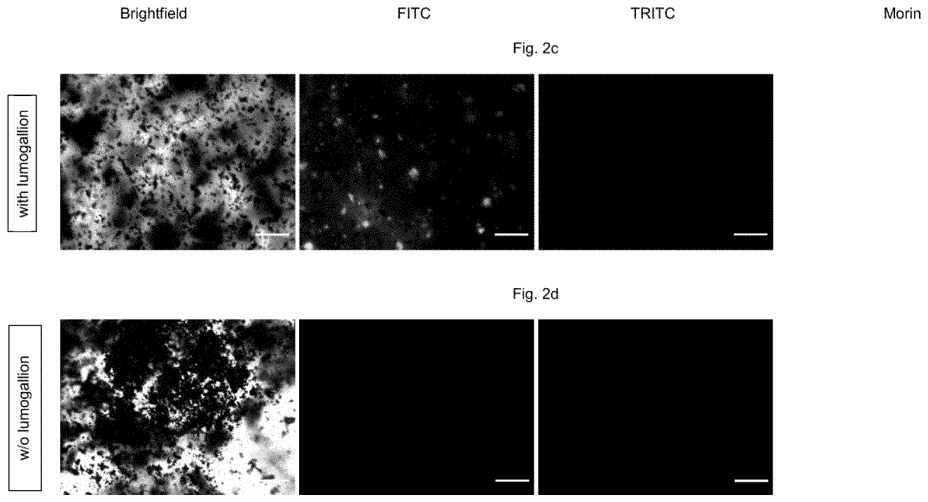

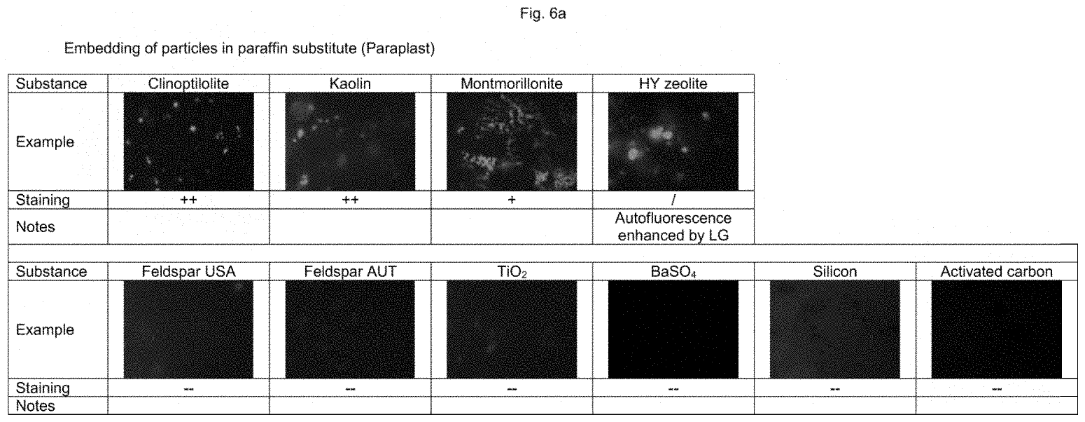

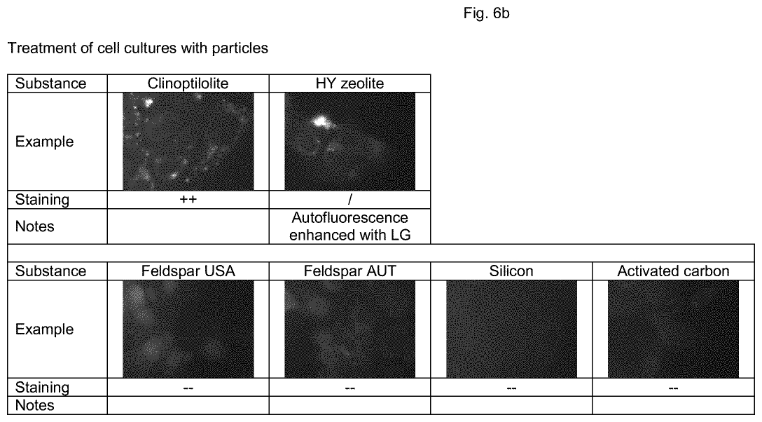

The labeling of aluminum in aluminosilicates not only allows the detection of zeolites in biological samples, but also to differentiate them in rock samples from other minerals using lumogallion staining—albeit surprisingly only after embedding them in paraffin/paraffin substitute. This embedding turns out to be a decisive and unavoidable step, as well as a step never described so far in the detection process (see FIG. 2), without which there is no evaluable labeling with lumogallion (see FIG. 3). A summary of examples of various rocks showing staining or no staining with lumogallion can be found in FIG. 6a.
The staining of aluminum with lumogallion offers the advantage of being feasible under physiological pH (7.0-7.2) and using conventional (cell culture) buffers (such as HEPES or PIPES) and media. Alternatively, the specimens may also be incubated in the acidic range and with other conventional buffers such as 20 mM acetate buffer, pH 4.0. The wide range of buffers and pH values is extremely useful in practical applications.
To detect fluorescence resulting from the reaction of lumogallion with aluminum from the sample, no special equipment dedicated to this purpose is necessary, but commercially available epifluorescence and confocal microscopes and spectrophotometers with the appropriate filters can be used.
Due to the relatively high stability of the labeling after fixation and embedding using a mounting medium, the samples can be examined or stored under a microscope for longer periods of time (several days to months) without significant loss of quality, which is particularly characteristic of the invention (see e.g. FIG. 5).

The invention will be described in more detail below, wherein the individual Figures show the following:

FIG. 1—Lumogallion staining of zeolite in fixed cell cultures

Evaluation of the samples was carried out on an epifluorescence microscope using special filters (see Table 2).

The excitation took place at about 500 nm, the emission was detected at around 570 nm.

FIG. 2—Joint embedding of different particles (zeolite and activated carbon or zeolite and silicon) followed by lumogallion staining

Joint embedding of different rock samples showing either fluorescence staining or no fluorescence staining with lumogallion.

Both the specificity of lumogallion labeling for aluminum-containing compounds as well as the specificity of the individual filters for different rocks become apparent.

FIG. 3—Particle staining with lumogallion without prior embedding

Staining of zeolite particles was carried out without prior embedding in paraffin/paraffin substitute.

No labeling could be detected in the sample.

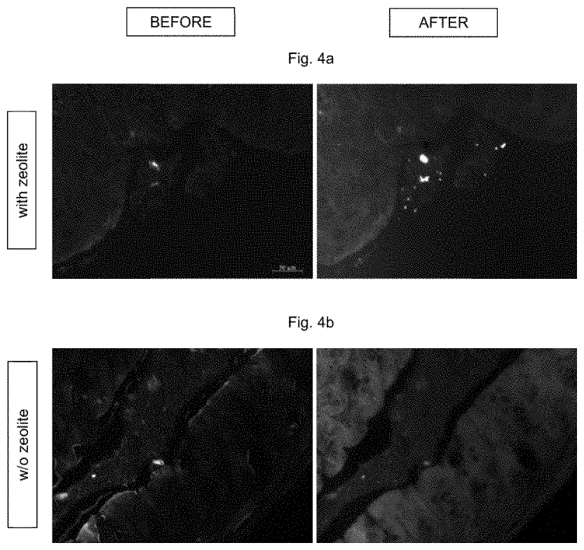

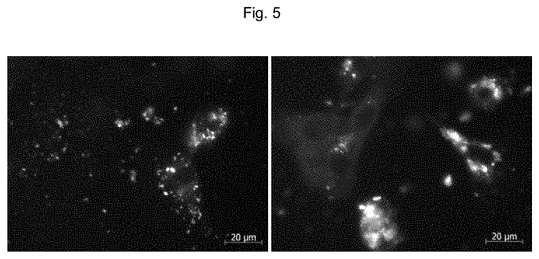

FIG. 4—Lumogallion-stained histological samples

Paraffin-embedded, non-dewaxed intestinal tissue samples from mice fed with and without zeolite admixture in the diet after staining with lumogallion.

FIG. 5—Zeolite-treated, fixed and lumogallion-labeled cells which had been stored for 8 weeks at 4° C.

After the first analysis, the samples were kept unchanged in the refrigerator at 4° C. for 56 days before being re-evaluated.

FIG. 6—Fluorescence images of the test substances summarized in Table 1

Overview of various rock specimens embedded in paraffin substitute (Paraplast) or used for cell culture treatments, which stained positively with Lumogallion or could not be labeled with Lumogallion.

In FIGS. 1-5, clinoptilolite (“purified” according to U.S. Pat. No. 8,173,101 B2) was used; this is an aluminosilicate of volcanic origin and belongs to the group of zeolites within the tectosilicates.

FIG. 6 allows a comparison of clinoptilolite with the artificially produced HY zeolite both when used in cell cultures and after embedding in paraffin substitute (Paraplast).

In detail, the figures represent:

FIG. 1 shows a typical analysis with an epifluorescence microscope with 100× magnification (Axiovert 200M, ZEISS company) of an application of the lumogallion staining of paraformaldehyde-fixed human cell cultures (MCF-10A, ATCC CRL-10317) with (FIG. 1a) and without (FIG. 1b) clinoptilolite treatment. Clinoptilolite is an aluminosilicate of volcanic origin and belongs to the group of zeolites within the tectosilicates. Prior to this, the samples were incubated overnight at 37° C. with 100 μM lumogallion in 50 mM HEPES buffer, pH 7.2. In order to minimize fading of fluorescence staining, an antioxidant mounting medium (VECTASHIELD Antifade Mounting Medium, Vector Laboratories, USA) was used. The filters described in Table 2 were used for the analysis. Clinoptilolite particles fluoresce clearly and—using the FITC filter (FIG. 1a)—can even be recognized individually. The untreated clinoptilolite particles of the control show no autofluorescence (FIG. 1b). The scale bar corresponds to 20 μm.

FIG. 2 depicts joint embedding of clinoptilolite with particles which are not stainable with lumogallion. For this purpose, the particles (clinoptilolite and activated carbon, FIGS. 2a and 2b, or clinoptilolite and silicon, FIGS. 2c and 2d) were embedded in paraffin substitute (Paraplast X-tra Tissue Infiltration/Embedding Medium, McCormick Scientific, PA) and subsequently Mictrotome-sectioned at room temperature with a specimen thickness of 16 μm. For staining with 100 μM lumogallion in 50 mM HEPES buffer, pH 7.2, the section was incubated overnight at 37° C. (FIGS. 2a and 2c). FIGS. 2b and 2d show the associated controls which were treated the same as the samples—except that they were incubated overnight in 50 mM HEPES buffer without lumogallion. Both controls and samples were protected from bleaching with mounting medium (VECTASHIELD Antifade Mounting Medium, Vector Laboratories, USA). The samples used in FIGS. 2a and 2b as well as 2c and 2d are each immediately successive serial sections with different areas shown in the figures. As can be clearly seen in FIGS. 2a and 2c, an evaluation using an epifluorescence microscope with 100× magnification (Axiovert 200M, ZEISS), and in the case of the activated carbon-clinoptilolite mixture an evaluation with all the filters described in Table 2, is possible without difficulty—the staining of clinoptilolite is vibrant while the activated carbon does not fluoresce. The situation was similar with a barium sulfate-clinoptilolite mixture (not shown). However, the silicon-clinoptilolite mixture could only be analyzed with the FITC and TRITC filters, since the excitation in the UV light range required for evaluation using morin filters heated the silicon particles, causing the Paraplast to melt and the particles to begin to diffuse, which made detection impossible. This impressively illustrates the high specificity of the labeling of aluminum using lumogallion, as well as the need to use suitable filters which are to be chosen specifically for the individual samples. The scale bar corresponds to 20 μm.

FIG. 3 represents particle staining of clinoptilolite without prior embedding in paraffin or Paraplast in brightfield and using different filters (FITC, TRITC, Morin). The samples were incubated with a particle concentration of 133 μg/ml with 100 μM lumogallion in 50 mM HEPES buffer, pH 7.2 at 37° C. overnight, then centrifuged, and the pellet was fixed in 15 μl mounting medium (VECTASHIELD Antifade Mounting Medium, Vector Laboratories, USA) on a microscope slide. Labeling of the clinoptilolite particles with lumogallion is hardly detectable with an epifluorescence microscope with 100× magnification (Axiovert 200M, ZEISS) and therefore can not be evaluated. The scale bar corresponds to 20 μm.

FIG. 4 illustrates murine, non-dewaxed, histological intestinal samples of a clinoptilolite-fed mouse (FIG. 4a) and a control mouse without clinoptilolite admixture in the feed (FIG. 4b). Tissue sections were imaged with a 32× magnification epifluorescence microscope (Axiovert 200M, ZEISS), first untreated (FIGS. 4a and 4b “BEFORE”) and subsequently stained with lumogallion (FIGS. 4a and 4b “AFTER”). The scale bar corresponds to 50 μm. After sampling, fixation, and subsequent paraffin embedding, the samples were stored at room temperature for about 2.5 years before being sectioned (to approximately 15 μm) and used for lumogallion staining. The sections were incubated overnight at 37° C. with 100 μM lumogallion in 50 mM HEPES buffer, pH 7.2, and then fixed on a microscope slide using mounting medium (VECTASHIELD Antifade Mounting Medium, Vector Laboratories, USA). The staining of the particles is clearly visible (FIG. 4a “AFTER”). The long storage period of histological sections did not minimize the quality of lumogallion labeling of clinoptilolite particles. FIG. 4b “AFTER” illustrates the quenching of autofluorescence by incubation with lumogallion.

FIG. 5 shows two examples of fluorescence images of an evaluation of samples labeled with lumogallion after a longer storage period (8 weeks at 4° C.). Human cell cultures (MCF-10A, ATCC CRL-10317) were fixed with paraformaldehyde following clinoptilolite treatment and subsequently incubated with lumogallion. After initial analysis, the clear-coated samples remained unopened for 2 months under continuous cooling without further manipulation, before additional analysis (FIG. 5) was performed. Evaluation was carried out at 100× magnification (Axiovert 200M, ZEISS) using the FITC filter. Clinoptilolite particles continue to fluoresce intensely—fluorescence does not appear to be noticeably reduced due to the long storage period. 100× magnification. The scale bar corresponds to 20 μm.

FIG. 6 shows a summary of the test substances used and their reaction to lumogallion incubation. While FIG. 6a shows the particles embedded in Paraplast (paraffin substitute), FIG. 6b summarizes cell cultures incubated with aluminosilicate or non-aluminosilicate particles, then fixed and finally treated with lumogallion. The samples were always evaluated under the same conditions (FITC/TRITC/morin filter, Axiovert 200M, ZEISS) and using the same magnification (100×). The intensity of the staining is indicated as follows: “++” means strong, “+” means weak and “−−” means no labeling of the particles with lumogallion. The symbol “/” was used to indicate experimental problems, which are further explained in the “Notes” column; in the examples given, it specifically was a strong autofluorescence of the test substance.

Specific detection of aluminosilicate compounds in cells (FIGS. 1, 5, and 6b) and tissues (FIG. 4) enables the identification and distribution of the same, and thus for the first time, detection and tracking of these particles after their application. Of great advantage is the possibility of subsequent labeling of aluminosilicates in biological material, since the lumogallion reaction is also feasible in paraformaldehyde-fixed preparations without loss of quality of the label. Dewaxing of samples embedded in paraffin/paraffin substitute is surprisingly unnecessary for successful staining.

Furthermore, staining with lumogallion surprisingly quenches any autofluorescence of the cells or the cell structure (FIG. 4b “AFTER”), which further contributes to a distinct identification of the zeolite particles.

Labeling of the particles surprisingly proves to be very stable, so that these are clearly visible even days or months later (FIG. 5).

The ability to label zeolite particles over a broad temperature range and at variable pH values significantly expands the application spectrum and contributes to a diverse application.

Staining of aluminosilicate particles with only lumogallion is also possible (FIG. 6a)—but surprisingly only if these were previously embedded in paraffin or paraffin substitute (see FIG. 3, which shows no staining of particles with lumogallion without paraffin or Paraplast). As a result, zeolites can also be distinguished from other substances or minerals—examples being joint embedding of clinoptilolite and activated carbon (FIGS. 2a and 2b), or clinoptilolite and silicon (FIGS. 2c and 2d), or also clinoptilolite and barium sulfate (not shown), wherein in each case only clinoptilolite was stained, while activated carbon (FIG. 2a), silicon (FIG. 2c), and barium sulfate respectively remained non-fluorescent.

If necessary, re-staining of bleached particles is possible without restriction and useful, if previously labeled samples need to be analyzed again. To this end, the samples are briefly rinsed three times in the buffer in which the subsequently used lumogallion solution was prepared (e.g., HEPES or acetate buffer) and then incubated in the lumogallion labeling solution. Incubation time and temperature, as well as the further procedure up to and including embedding may correspond to those of the primary incubation (see embodiments).

In addition, labeling with lumogallion has the advantage of being detectable over a broad spectrum so that the most common filters (see Table 2) may be used. In this case, it is recommended to test the filter most suitable for each respective test series—minerals or rocks have different fluorescence maxima/fluorescence spectra with lumogallion staining and the choice of filters should be taken into consideration in this regard (for example FIG. 2c—in this case, silicon particles cannot be detected with a morin filter).

The user can work with the usual equipment of a cell biological laboratory; the acquisition of specialized equipment produced solely for lumogallion staining is unnecessary.

In summary, the invention is therefore characterized by its rapid and uncomplicated practicability, its specificity for aluminosilicates, its high reproducibility and stability over several days or months, as well as by the elimination of dewaxing of histological specimens and the possibility of renewed/repeated staining with lumogallion.

This characteristic may prove particularly useful in forensic analysis of soil samples (Tibet and Carter, 2008). It is also possible to identify aluminosilicates in human and animal histospecimens which were created in the distant past and may thus yield new insights—without additional larger investments of money and time.

Furthermore, it is possible to identify aluminosilicates in mineral mixtures/rock mixtures in an equally specific manner and also, as a consequence, to define the amount of aluminosilicates in the substance to be tested.

Within a short period of time and with little technical resources and required staff, this method achieves a highly reproducible detection of aluminosilicates in the context of analytical-diagnostic quick detection or for visualization using fluorescence microscopy and the appropriate filter.

### Examples of Embodiments

### Example of Labeling of Aluminum in Cell Culture Samples:

### Sample Preparation:

Various rock samples (see Table 1) were diluted in growth medium with antibiotics (penicillin/streptomycin) to a final concentration of C_{E}≙≈100 mg/ml.
This suspension served as stock solution and was stored at 4° C.

### Test Procedure:

A defined period of time before the start of the experiment, cells of a selected culture (for example, one day before the start of the experiment if MCF-10A cells, ATCC CRL-10317, were used; or 5 days in the case of Caco-2 cells, ATCC HTB-34) were seeded on sterile round glass slides (diameter 10 mm) in petri dishes.
After 24 hours under growth conditions, the cells (1 glass slide per batch) were incubated with 10 μg/cm² of one of the test substances (see Table 1) under growth conditions for a further 24 hours.
The glass slides were then washed once with 50 mM HEPES buffer, pH 7.2, and fixed at room temperature in 0.5%-2.0% paraformaldehyde in 50 mM HEPES buffer, pH 7.2, for 30 min.
Afterwards, the glass slides were again washed 3× in the above HEPES buffer before being incubated protected from light in 1 ml of
   a) 100 μM lumogallion solution in 50 mM HEPES buffer, pH 7.2,
      or
   b) 200 μM lumogallion solution in 20 mM acetate buffer, pH 4.0,
for either
   2 to 4 hours at 60° C. to 80° C.,
   or
   12 to 24 hours at room temperature to 37° C.
Prior to embedding in mounting medium (VECTASHIELD Antifade Mounting Medium, Vector Laboratories, USA), the glass slides were briefly dipped in double-distilled water and drained on a clean paper towel.
The embedded samples were attached to the slide in an airtight manner using a viscous clear coat to prevent desiccation.
The individual samples were then assayed for lumogallion labeling using an epifluorescence microscope and various filters (TRITC, FITC, or morin filters, see Table 2) (see FIGS. 1 and 6b).

### Example of Labeling of Aluminosilicates in Histosections:

### Test Procedure for Dewaxed Samples:

1. The tissue samples embedded in paraffin or paraffin substitute (Paraplast) were sectioned on the cryomicrotome to a specimen thickness of 5 μm to 25 μM and transferred to glass slides.
2. Afterwards, the sections were washed 3× with PBS.
3. This was followed by incubation in Neo-Clear (xylene substitute for microscopy, Merck, Germany) for 3 min under gentle shaking.
4. Then the used Neo-Clear was removed and replaced with fresh Neo-Clear, which in turn remained on the sample under gentle shaking. This incubation lasted 1 min.
5. Afterwards, the section was subjected to several washing steps:
   3× with PBS
   3× with EtOH_{absolut}
   3× with 50 mM Hepes, pH 7.2,
6. Followed by incubation with
   a) 100 μM lumogallion in 50 mM HEPES buffer, pH 7.2,
      or
   b) 200 μM lumogallion in 20 mM acetate buffer, pH 4.0,
   for 12 to 24 hours (“overnight”) at 37° C.
7. In order to achieve a longer shelf life of the fluorescence staining, the sections were covered with mounting medium (for example VECTASHIELD Antifade Mounting Medium, Vector Laboratories, USA) and covered with a cover slip.
8. The embedded sections were protected from desiccation by attaching the cover slip to the slide using a viscous clear coat.
9. The individual samples were then assayed for lumogallion labeling using a fluorescence microscope and various filters (TRITC, FITC, or morin filters, see Table 2).
   Test Procedure for Samples Coated with Paraffin:

Paraffin sections were prepared as described in the above protocol (Item 1), washed on the slides (Item 5), incubated with lumogallion (Item 6), embedded (Items 7 and 8), and analyzed using epifluorescence microscopy (Item 9). Items 2, 3, and 4 were omitted. FIG. 4 will be used as an example of the application.

### Example of Labeling of Aluminosilicates in Mineralogical-Geological Samples:

1. The glass slides were cleaned with 70% ethanol to allow adhesion of the liquid blocker super PAP pen (Liquid-Repellent Slide Marker Pen, Science Services, Germany). It was used to draw rectangles on the glass, which after drying were used as “tubs” for filling with buffer or lumogallion solution.
2. The rock samples were ground to fine particles (approximately 1 μm to 60 μm in diameter) (see Table 1) and embedded in paraffin substitute (Paraplast X-Tra Tissue Infiltration/Embedding Medium, McCormick Scientific, PA), cut to a specimen thickness of 14 μm to 16 μm (similar to that of the tissue samples) on the cryomicrotome before being transferred to the previously prepared glass slides.
3. The sections floating in the buffer or lumogallion solution were incubated at 37° C. for 12-24 hours.
4. Afterwards, they were applied to new slides, overlaid with VECTASHIELD Antifade Mounting Medium (Vector Laboratories, USA) and covered with a cover slip.
5. The edges between the slides and cover slips were sealed with transparent clearcoat.
6. After the clearcoat dried, the samples were examined using epifluorescence microscopy (see FIG. 6a—individual substances, and FIG. 2—mixtures). For this, the various filters described in Table 2 (TRITC, FITC, or morin filters) were used.

TABLE 1
Table 1: Test substances used (selection)
and their manufacturers or distributors.

| Substance | Product lnformation/(Product Number) |
|---|---|
| Activated carbon | Activated carbon, pure |
| | Merck (102183) |
| Aluminum oxide | Aluminum oxide |
| | Sigma-Aldrich (342750) |
| Barium sulfate | Prepared from |
| | Barium chloride dihydrate 99.995% Suprapur |
| | Sigma-Aldrich (101716) |
| | and |
| | Anhydrous sodium sulfate for analysis ACS, |
| | ISO, Reag. Ph Eur Merck (106649) |
| Calcium | Calcium carbonate, precipitated, analytical |
| carbonate | grade Ph.Eur., USP AppliChem (A0774) |
| Feldspar AUT | Feldspar, dry |
| | Amberger Kaolinwerke |
| | (FS900 SF Hirschau) |
| Feldspar USA | Feldspar Fortispar K-30 |
| | I Minerals Inc. |
| | (ULTRA HalloPure) |
| Halloysite | Halloysite |
| | I Minerals Inc. |
| | (ULTRA HalloPure) |
| Clinoptilolite | Glock Health, Science and Research |
| | (018-01-08-2-1-1) |
| | (Example of a natural zeolite whose heavy metal |
| | ions were exchanged for calcium according to |
| | U.S. Pat No. 8,173,101 B2.) |
| Kaolin | Kaolin chamotte |
| | Amberger Kaolinwerke |
| | (AS45 6.400 Hirschau) |
| Montmorillonite | Montmorillonite naturally occurring mineral |
| | Alfa Aesar/VWR (42531.22) |
| Silicon | Silicon powder, APS 1-5 micron, 99.9% (metals basis) |
| | Alfa Aesar/Thermo Fischer (Kandel) (44185) |
| Titanium dioxide | Titanium(IV)oxide, anatase |
| | Sigma-Aldrich (248576) |
| Zeolite HY | Zeolyst International |
| | (CBV400) |
| | (Example of an artificially synthesized zeolite) |

TABLE 2
Table 2: Fluorescence filters used

| | | |
|---|---|---|
| Filter | λ_{Absorption} [nm] | λ_{Emission} [nm] |

| | | | | |
|---|---|---|---|---|
| TRITC | 546/12 | (band pass) | 575-640 | (band pass) |
| | 560 | (beam splitter) | | |
| FITC | 450-490 | (band pass) | 515-565 | (band pass) |
| | 510 | (beam splitter) | | |
| Morin | 433/24 | (band pass) | >473 | (long pass) |
| | 465 | (beam splitter) | | |

Using a mounting medium and fluorescence microscopy and various filters (TRITC, FITC, morin), the samples prepared according to the invention can be stably detected and differentiated from a large number of non-aluminosilicates for a period of time ranging from days to several months. In cell biological and histological samples, which were embedded in either paraffin or paraffin substitute (Paraplast or the like), the samples can be labeled without the need for dewaxing using the method according to the invention.

Compared to the prior art, this constitutes a quick, simple, reliable, and cost-effective method.

As used in the description and claims, “substantially” means a deviation of up to 10% of the stated value, if this is physically possible, both downwards and upwards, otherwise only in the reasonable direction; indications in regard to temperatures are thus meant to be read as deviations of ±10° C.

All quantities and proportions, in particular those for delimiting the invention, as far as these do not relate to the specific examples, are to be understood with ±10% tolerance. Thus, for example: 11% means: from 9.9% to 12.1%. Percentages of ingredients are by weight unless otherwise specified. For terms such as “a solvent”, the word “a” is not to be regarded as a numerical word, but as a pronoun or as an indefinite article, unless the context indicates otherwise.

The term: “Combination” or “Combinations” means, unless otherwise indicated, all types of combinations, starting from two of the relevant constituents, to a multiplicity of such constituents, to all constituents. The term “comprising” also means “consisting of”.

The characteristics and variants specified in the individual embodiments and examples may be freely combined with those of the other examples and embodiments, and may in particular be used to characterize the invention in the claims without necessarily entraining the other details of the respective embodiment or the respective example.

### REFERENCES

### Non-Patent Literature (Shortened to the Most Important Publications)

### Babourina O, Rengel Z.

Uptake of aluminium into Arabidopsis root cells measured by fluorescent lifetime imaging.

### Ann Bot. 2009; 104(1): 189-195.

doi: 10.1093/aob/mcp098

### Caschetto S, Wollast R.

Dissolved aluminum in interstitial waters of recent marine sediments.

### Geochim Cosmochim Acta. 1979; 43: 425-428.

### Coons A H, Creech H J, Jones R N.

Immunological properties of an antibody containing a fluorescent group.

### Proc Soc Exp Biol Med. 1941; 47: 200-202.

### Coons A H, Creech H J, Jones R N, Berliner E.

The demonstration of pneumococcal antigen in tissues by the use of fluorescent antibody.

### J Immunol. 1942; 45: 159-170.

### Coons A H, Melvin H, Kaplan M H.

Localization of antigen in tissue cells: II. Improvements in a method for the detection of antigen by means of fluorescent antibody.

### J Exp Med. 1950; 91(1): 1-13.

### Cordes T, Maiser A, Steinhauer C, Schermelleh L, Tinnefeld P.

Mechanisms and advancement of antifading agents for fluorescence microscopy and single-molecule spectroscopy.

### Phys Chem Chem Phys. 2011; 13(14): 6699-6709.

doi: 10.1039/c0cp01919d

### Crompton T R.

Determination of metals in natural waters, sediments, and soils.
Elsevier, Amsterdam. 2015; 1^{st} Edition: 318 pages.

### Eticha D, Stass A, Horst W J.

Localization of aluminium in the maize root apex: can morin detect cell wall-bound aluminium?

### J Exp Bot. 2005; 56(415): 1351-1357.

doi:10.1093/jxb/eri136

### Florijn R J, Slats J, Tanke H J, Raap A K.

Analysis of antifading reagents for fluorescence microscopy.

### Cytometry. 1995; 19(2): 177-182.

doi: 10.1002/cyto.990190213

### He H-B, Lee H-K, Li S F Y, Hsieh A-K, Chi H, Siow K-S.

Determination of Trace Levels of Aluminum by Capillary Electrophoresis with Lumogallion Fluorometric Detection.

### J Chromatogr Sci. 1997; 35(7): 333-336.

doi: 10.1093/chromsci/35.7.333

### Huff J C, Weston W L, Wanda K D.

Enhancement of specific immunofluorescent findings with use of a para-phenylenediamine mounting buffer.

### J Invest Dermatol. 1982; 78(5): 449-450.

### Hydes D J, Liss P S.

The determination of low concentrations of dissolved aluminum in natural waters.

### Analyst. 1976; 101: 922-931.

### Illés P, Schlicht M, Pavlovkin J, Lichtscheidl I, Baluska F, Ovecka M.

Aluminium toxicity in plants: internalization of aluminium into cells of the transition zone in Arabidopsis root apices related to changes in plasma membrane potential, endosomal behaviour, and nitric oxide production.

### J Exp Bot. 2006; 57(15): 4201-4213.

doi: 10.1093/jxb/erl197

### Johnson G D, Nogueira Araujo G M.

A simple method of reducing the fading of immunofluorescence during microscopy.

### J Immunol Methods. 1981; 43: 349-350.

### Johnson G D, Davidson R S, McNamee K C, Russell G, Goodwin D, Holborow E J.

Fading of immunofluorescence during microscopy: a study of the phenomenon and its remedy.

### J Immunol Methods. 1982; 55(2): 231-242.

### Jonasson R G.

Investigation of the reactions of aquatic aluminum through fluorometry using lumogallion.
McMaster University, 1980. [http://hdl.handle.net/11375/18023].

### A Thesis Submitted to the Department of Geology in Partial Fulfillment of the Requirements for the Degree Bachelor of Science.

### Kataoka T, Iikura H, Nakanishi T M.

Aluminum distribution and viability of plant root and cultured cells.

### Soil Science and Plant Nutrition. 1997; 43: 1003-1007.

doi: 10.1080/00380768.1997. Ser. No. 11/863,707

### Klein J P, Mold M, Mery L, Cottier M, Exley C.

Aluminum content of human semen: implications for semen quality.

### Reprod Toxicol. 2014; 50: 43-48.

doi: 10.1016/j.reprotox.2014.10.001

### Lee B L, Chua L H, Ong H Y, Yang H G, Wu J, Ong C N.

Determination of serum and urinary aluminum by HPLC with fluorometric detection of Al-lumogallion complex.

### Clin Chem. 1996; 42(9): 1405-1411.

### Li Z, Xing F, Xing D.

Characterization of target site of aluminum phytotoxicity in photosynthetic electron transport by fluorescence techniques in tobacco leaves.

### Plant Cell Physiol. 2012; 53(7): 1295-1309.

doi: 10.1093/pcp/pcs076

### Longin A, Souchier C, Ffrench M, Bryon P A.

Comparison of anti-fading agents used in fluorescence microscopy: image analysis and laser confocal microscopy study.

### J Histochem Cytochem. 1993; 41(12): 1833-1840.

### Mile I, Svensson A, Darabi A, Mold M, Siesjö P, Eriksson H.

Al adjuvants can be tracked in viable cells by lumogallion staining.

### J Immunol Methods. 2015; 422: 87-94.

doi: 10.1016/j.jim.2015.04.008

### Ming D W, Dixon J B.

### Quantitative Determination of Clinoptilolite in Soils by a Cation-Exchange Capacity Method.

### Clays and Clay Minerals. 1987; 35: 463-468.

### Mirza A, King A, Troakes C, Exley C.

### The Identification of Aluminum in Human Brain Tissue Using Lumogallion and Fluorescence Microscopy.

### J Alzheimers Dis. 2016; 54(4): 1333-1338.

### doi: 10.3233/JAD-160648

### Mirza A, King A, Troakes C, Exley C.

Aluminium in brain tissue in familial Alzheimer's disease.

### J Trace Elem Med Biol. 2017; 40: 30-36.

doi: 10.1016/j.jtemb.2016.12.001

### Mold M, Eriksson H, Siesjö P, Darabi A, Shardlow E, Exley C.

Unequivocal identification of intracellular aluminium adjuvant in a monocytic THP-1 cell line.

### Sci Rep. 2014; 4: 6287.

doi: 10.1038/srep06287

### Mold M, Shardlow E, Exley C.

Insight into the cellular fate and toxicity of aluminium adjuvants used in clinically approved human vaccinations.

### Sci Rep. 2016; 6: 31578.

doi: 10.1038/srep31578

### Mumpton F A.

La roca magica: uses of natural zeolites in agriculture and industry.

### Proc Natl Acad Sci USA. 1999; 96(7): 3463-3470.

### Nishikawa Y, Hiraki K, Morishige, Shigematsu T.

Fluorophotometric determination of aluminum and gallium with lumogallion.

### Japan Analyst (Bunseki Kagaku). 1967; 16: 692-697.

### Nishikawa Y, Hiraki K, Morishige K, Tsuchiyama A, Shigematsu T.

Fluorometric determination of trace amount of aluminum in sea water.

### Japan Analyst (Bunseki Kagaku). 1968; 17: 1092-1097.

### Platt J L, Michael A F.

Retardation of fading and enhancement of intensity of immunofluorescence by p-phenylenediamine.

### J Histochem Cytochem. 1983; 31(6): 840-842.

doi: 10.1177/31.6.6341464

### Powell J J.

Analysis of aluminosilicate particles in biological matrices using histochemistry and X-ray microanalysis.

### Analyst. 2002; 127: 842-846.

### doi: 10.1039/B110761P

### Ramos Jareño M E.

Dissolution mechanism of montmorillonite in synthetic lung fluids: effect of organic ligands and biodurability.
Doctoral dissertation.

### Granada: Universidad de Granada, 2013. 294 p.

Retrieved from http://digibug.ugr.es/handle/10481/29552#.WYLoR7mQqHs.

### Ren J L, Zhang J, Luo J Q, Pei X K, Jiang Z X.

Improved fluorimetric determination of dissolved aluminium by micelle-enhanced lumogallion complex in natural waters.

### Analyst. 2001; 126(5): 698-702.

doi: 10.1039/b007593k

### Shigematsu T, Nishikawa Y, Hiraki K, Nagano N.

Fluorometric determination of trace amount of aluminum in natural water by lumogallion method. Masking of ferric iron with o-phenanthroline.

### Japan Analyst (Bunseki Kagaku). 1970; 19(4): 551-554.

### Sivaguru M, Liu J, Kochian L V.

Targeted expression of SbMATE in the root distal transition zone is responsible for sorghum aluminum resistance.

### Plant J. 2013; 76(2): 297-307.

doi: 10.1111/tpj.12290

### Sposito G.

The environmental chemistry of aluminum.
CRC Press, Boca Raton. 1995; 2^{nd} Edition: 480 pages.

### Sutheimer S H, Maurice P A, Zhou Q.

Dissolution of well and poorly crystallized kaolinites; Al speciation and effects of surface characteristics.

### American Mineralogist. 1999; 84(4): 620-628.

doi: 10.2138/am-1999-0415

### Tibbett M, Carter D O.

### Soil Analysis in Forensic Taphonomy: Chemical and Biological Effects of Buried Human Remains.

CRC Press. Feb. 27, 2008; 364 pages.

### Uchiumi A, Takatsu A, Teraki Y.

Sensitive detection of trace aluminium in biological tissues by confocal laser scanning microscopy after staining with lumogallion.

### Analyst. 1998; 123(4): 759-762.

### Valnes K, Brandtzaeg P.

Retardation of immunofluorescence fading during microscopy.

### J Histochem Cytochem. 1985; 33(8): 755-761.

doi:10.1177/33.8.3926864

### Yamada E, Hiwada T, Inaba T, Tokukura M, Fuse Y.

Speciation of aluminum in soil extracts using cation and anion exchangers followed by a flow-injection system with fluorescence detection using lumogallion.

### Anal Sci. 2002; 18(7): 785-791.

### Zhou Y, Yokel R A.

The chemical species of aluminum influences its paracellular flux across and uptake into Caco-2 cells, a model of gastrointestinal absorption.

### Toxicol Sci. 2005; 87(1): 15-26.

doi: 10.1093/toxsci/kfi216

### Patent Literature

Anti-counterfeit penetrant atomic stamp pad ink and its preparation method, 1996
CN 96104762
Anti-fake fluorescent pressure-sensitive carbon paper generating invisible duplication handwriting and making method thereof, 1996
CN 96100340
Anti-fake fluorescent pressure-sensitive carbon paper, 1996
CN 96100339
Changed condition indicator, 2001
U.S. Ser. No. 11/375,307
Chelate reagent and measuring aluminum and measuring method, 2000
U.S. Ser. No. 10/380,242
Detector used for investigating water consists of an active ingredient coating formed on a carrier, 2000
DE2000160845
Diagnostic test for elemental imbalances, 2002
PCT/US2003/012911 and EP20030719936
Dye lasers, 1978
U.S. Ser. No. 05/963,157
Fiber optic moisture sensor, 1992
U.S. Ser. No. 07/914,795 and U.S. Ser. No. 08/201,820
Fluorescent anti-fake mark and its making method, 1994
CN 94105055
Fluorescent probe for the detection of corrosion, 2005
PCT/G B2006/001626
Method for analyzing trace quantity of Aluminum, 2003
JP20020104985 20020408
Method for determination of aluminum, 2001
U.S. Ser. No. 10/498,060
Methods of monitoring rinsing solutions and related systems and reagents, 2004
U.S. Ser. No. 10/952,510
Method of producing crosslinkable silyl group-containing polyoxyalkylene polymers, 2000
U.S. Ser. No. 09/832,344 and EP20010109302
Organic electroluminescence device, 2000
U.S. Ser. No. 09/915,452
Organic LED element, 1999
JP19980083987 19980330
pH-sensitive microparticles with matrix-dispersed active agent, 2011
U.S. Ser. No. 13/542,155
Photodeposition method for fabricating a three-dimensional, patterned polymer microstructure, 1995
U.S. Ser. No. 08/519,062
Semiconductor device and adhesive sheet, 2009
CN 201010135730
Semiconductor wafer cleaning agent and cleaning method, 2000
U.S. Ser. No. 10/203,659
Synergistic combinations of chromate-free corrosion inhibitors, 2005
PCT/US2006/007305 and U.S. Ser. No. 11/817,659
Verfahren zur Herstellung vernetzbarer Silylgruppen enthaltender Polyoxyalkylenpolymere, 2000
DE2001625268

The content of the English language references, especially of the patent literature, is incorporated herein by reference for the jurisdictions in which this is possible.

1-7. (canceled)

8. A method for the specific fluorescence labeling of aluminosilicates in a sample, comprising:
incubating the sample with a lumogallion (5-chloro-3-(2,4-dihydroxyphenylazo)-2-hydroxybenzenesulfonic acid) solution in a buffer having a pH value ranging from pH 7.2 to pH 4.0; wherein incubating the sample includes either
incubating the sample for a period of from 2 to 4 hours at a temperature of 60 to 80° C., or
incubated the sample for a period of time from 12 to 24 hours at a temperature of 20 to 30° C.; and
stabilizing the sample using a mounting medium.

9. The method of claim 8, wherein the specific fluorescence labeling of aluminosilicates in the sample includes the specific fluorescence labeling of zeolites in the sample.

10. The method of claim 8, wherein the specific fluorescence labeling of aluminosilicates in the sample includes the specific fluorescence labeling of clinoptilolites purified from heavy metals in the sample.

11. The method of claim 8, wherein the specific fluorescence labeling of aluminosilicates in the sample includes specific fluorescence labeling of aluminosilicates in a biological sample.

12. The method of claim 8, wherein the specific fluorescence labeling of aluminosilicates in the sample includes specific fluorescence labeling of aluminosilicates in a cell sample.

13. The method of claim 8, wherein the specific fluorescence labeling of aluminosilicates in the sample includes specific fluorescence labeling of aluminosilicates in a histological sample.

14. The method of claim 8, wherein the specific fluorescence labeling of aluminosilicates in the sample includes specific fluorescence labeling of aluminosilicates in a sample embedded in either a paraffin or a paraffin substitute.

15. The method of claim 14, wherein labeling the sample includes labeling the sample without dewaxing the sample.

16. The method of claim 8, wherein the specific fluorescence labeling of aluminosilicates in the sample includes specific fluorescence labeling of aluminosilicates in a mineralogical sample or a geological sample, and where the sample was embedded in either a paraffin or a paraffin substitute.

17. The method of claim 8, wherein the specific fluorescence labeling of aluminosilicates in the sample includes specific fluorescence labeling of aluminosilicates in an in vitro-cell culture sample; and further comprises
incubating the in vitro-cell culture sample with aluminosilicates or with mixtures of aluminosilicates and non-aluminosilicates; and
fixing the sample;
before treating the fixed sample with the lumogallion solution.

18. The method of claim 8, wherein the specific fluorescence labeling of aluminosilicates in the sample further comprises an analytical-diagnostic rapid detection of the labeled sample, or an imaging of the sample.

19. The method of claim 8, wherein stabilizing the sample using a mounting medium includes stabilizing the sample using a mounting medium that includes para-phenylenediamine (1,4-diaminobenzene) in glycerol.
